(19) United States
(12) Patent Application Publication
(10) Pub. No.: US 2026/0250227 A1
(43) Pub. Date: Aug. 27, 2026

(54) STABILIZATION OF HIGH-CONCENTRATION FORMALDEHYDE SOLUTIONS

(71) Applicant: Celanese International Corporation, Irving, TX (US)

(72) Inventors: John Leslie Ray, JR., Kingsville, TX (US); Kenneth Allen Windhorst, Portland, TX (US); Michael Yearwood, Bishop, TX (US); Jose Mario Alaniz, JR., Corpus Christi, TX (US)

(73) Assignee: Celanese International Corporation, Irving, TX (US)

(21) Appl. No.: 19/061,460

(22) Filed: Feb. 24, 2025

(51) Int. Cl. C07C 45/86; C07C 45/29

(52) U.S. Cl. CPC C07C 45/86; C07C 45/29

(57) ABSTRACT

Free radical scavengers may be useful as stabilizers for aqueous formaldehyde solutions. For example, an aqueous formaldehyde solution may include 40 wt % or greater of water, 30 wt % or greater of formaldehyde, and 250 ppm or less of a free radical scavenger, each concentration based on a total weight of the aqueous formaldehyde solution.

### FIELD OF THE INVENTION

The present disclosure relates generally to compositions and methods for stabilizing high-concentration formaldehyde solutions and mitigating the formation of paraformaldehyde therein.

### BACKGROUND OF THE INVENTION

Formaldehyde is one of the most important commodity chemicals and is used in the production of various resins and polymers. Formaldehyde can be synthesized by partial oxidation of methanol in the presence of a catalyst. High concentration aqueous solutions of formaldehyde (e.g., greater than 30 wt %, especially greater than 50 wt %) may reduce shipping expense and reduce the volume of fluid to be handled and stored. However, at high concentrations, the solutions may become cloudy over time with the formation of paraformaldehyde as a result of formaldehyde self-polymerizing and off-test due to increased acidity. The self-polymerization may be mitigated by increasing the temperature of the solution, including stabilizers like methanol, or a combination thereof.

At very high concentrations of formaldehyde (e.g., greater than 50 wt %), the aqueous formaldehyde solution may be stored at 140° F. or greater and include 10 wt % or greater of methanol. However, at such high temperatures and concentrations of methanol, appreciable amounts of formic acid may form during storage within days to weeks. The formic acid may then facilitate reactions between formaldehyde and methanol to produce poly(oxymethylene) dimethyl ethers (POMDMEs). Accordingly, depending on the formaldehyde concentration and storage temperature, high concentration formaldehyde solutions may go out of the required purity specifications within weeks if not days. Processes like ion exchange treatment may be used to purify the out-of-specification formaldehyde solutions but may also produce high volumes of waste including aqueous solutions of sodium hydroxide and increase the cost of the final product. There is a need for stabilizing formaldehyde solutions, especially at high formaldehyde concentrations, for longer and at lower temperatures.

### SUMMARY OF THE INVENTION

Embodiments of the present disclosure may include an aqueous formaldehyde solution comprising: 40 wt % or greater of water; 30 wt % or greater of formaldehyde; and 250 ppm or less of a free radical scavenger, each concentration based on a total weight of the aqueous formaldehyde solution.

Embodiments of the present disclosure may include a method comprising: producing a product comprising formaldehyde from methanol; treating the product to produce an aqueous formaldehyde solution comprising 40 wt % or greater of water and 30 wt % or greater of formaldehyde, each concentration based on a total weight of the aqueous formaldehyde solution; and adding a free radical scavenger to the product, the aqueous formaldehyde solution, any intermediate fluid therebetween, or any combination thereof such that a concentration of the free radical scavenger in the aqueous formaldehyde solution is 250 ppm or less, based on the total weight of the aqueous formaldehyde solution.

Embodiments of the present disclosure may include a method comprising: storing an aqueous formaldehyde solution at 80° F. or greater, the aqueous formaldehyde solution comprising 40 wt % or greater of water, 30 wt % or greater of formaldehyde, and 250 ppm or less of a free radical scavenger, each concentration based on a total weight of the aqueous formaldehyde solution.

### BRIEF DESCRIPTION OF THE DRAWINGS

FIG. 1 is a plot of the concentration of formic acid over time for a control sample and a sample containing a free radical scavenger according to at least some embodiments of the present disclosure.

FIG. 2 is a plot of the concentration of formaldehyde over time for a control sample and a containing a free radical scavenger sample according to at least some embodiments of the present disclosure.

FIG. 3 is a plot of the concentration of formic acid over time for a control sample and a sample containing a free radical scavenger according to at least some embodiments of the present disclosure.

FIG. 4 is a plot of the concentration of formic acid over time for a control sample and a sample containing a free radical scavenger according to at least some embodiments of the present disclosure.

FIG. 5 is a plot of the concentration of formic acid over time for a control sample and a sample containing a free radical scavenger according to at least some embodiments of the present disclosure.

FIG. 6 is a plot of the concentration of formic acid over time for a control sample and a sample that includes a free radical initiator.

### DETAILED DESCRIPTION OF THE INVENTION

Described herein are compositions and methods for stabilizing high-concentration formaldehyde solutions and mitigating the formation of paraformaldehyde and formic acid therein. Without being limited by theory, the Cannizzaro reaction of formaldehyde with itself under acidic conditions may be facilitated by free radicals. The aqueous formaldehyde solutions of the present disclosure may include a free radical scavenger as a stabilizer to mitigate said reaction.

Advantageously the free radical scavenger may be at a low concentration compared to other stabilizers. For example, methanol may conventionally be used as a stabilizer, depending on the formaldehyde concentration, in amounts of at least 8 wt %, based on a total weight of the aqueous formaldehyde solution. In contrast, the free radical scavengers may be present in the aqueous formaldehyde solutions of the present disclosure at 250 ppm (i.e., 0.025 wt %) or less.

The inclusion of this additional stabilizer may allow for longer-term storage of aqueous formaldehyde solutions at high temperature or short-term storage of aqueous formaldehyde solutions at lower temperatures. Further, the inclusion of the free radical scavenger may allow for reducing the concentration of other stabilizers like methanol, if not eliminating the need for such stabilizers, depending on the formaldehyde concentration and storage temperature.

### Definitions and Descriptions

As used herein, the terms “invention,” “the invention,” “this invention” and “the present invention” are intended to refer broadly to all of the subject matter of this patent application and the claims below. Statements containing these terms should be understood not to limit the subject matter described herein or to limit the meaning or scope of the patent claims below.

As used herein, the meaning of “a,” “an,” or “the” includes singular and plural references, e.g., meaning “one or more,” unless the context clearly dictates otherwise.

All ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10.

As used herein, the term “comprises” and variations thereof is open-ended, but for purposes of the specification and as support for future claim amendments, it should be understood as alternatively disclosing more restrictive language, such as “consisting essentially of” or “consisting of.”

### Aqueous Formaldehyde Solutions

Aqueous formaldehyde solutions of the present disclosure may include water, formaldehyde, and a free radical scavenger.

Water may be present in the aqueous formaldehyde solutions of the present disclosure at 40 wt % or greater (e.g., 50 wt % or greater, 40 wt % to 70 wt %, 30 wt % to 45 wt %, 35 wt % to 50 wt %, or 40 wt % to 60 wt %), based on the total weight of the aqueous formaldehyde solution.

Formaldehyde may be present in the aqueous formaldehyde solutions of the present disclosure at 30 wt % or greater (e.g., 40 wt % or greater, 50 wt % or greater, 30 wt % to 60 wt %, 30 wt % to 45 wt %, 35 wt % to 50 wt %, or 40 wt % to 60 wt %), based on the total weight of the aqueous formaldehyde solution.

Examples of free radical scavengers may include, but are not limited to, 2,2,6,6-tetramethyl-1-piperidiny-1-oxyl (TEMPO), 4-hydroxy-2,2,6,6-tetramethyl-1-piperidin-1-oxyl (TEMPOL), a sterically hindered phenolic antioxidant, copper, 3,7-bis(dimethylamino)-phenothiazin-5-ium chloride (methylene blue), pentylenetetrazol (PTZ), the like, and any combination thereof.

Examples of sterically hindered phenolic antioxidants may include, but are not limited to, calcium bis(ethyl 3,5-di-tert-butyl-4-hydroxybenzylphosphonate) (IRGANOX® 1425); terephthalic acid, 1,4-dithio-, S,S-bis(4-tert-butyl-3-hydroxy-2,6-dimethylbenzyl) ester (CYANOX® 1729); triethylene glycol bis(3-tert-butyl-4-hydroxy-5-methylhydrocinnamate); hexamethylene bis(3,5-di-tert-butyl-4-hydroxyhydrocinnamate (IRGANOX® 259); 1,2-bis(3,5,di-tert-butyl-4-hydroxyhydrocinnamoyl)hydrazide (IRGANOX® 1024); 4,4′-di-tert-octyldiphenamine (NAUGALUBE® 438R); phosphonic acid, (3,5-di-tert-butyl-4-hydroxybenzyl)-, dioctadecyl ester (IRGANOX® 1093); 1,3,5-trimethyl-2,4,6-tris(3′,5′-di-tert-butyl-4′ hydroxybenzyl)benzene (IRGANOX® 1330); 2,4-bis(octylthio)-6-(4-hydroxy-3,5-di-tert-butylanilino)-1,3,5-triazine (IRGANOX® 565); isooctyl 3-(3,5-di-tert-butyl-4-hydroxyphenyl)propionate (IRGANOX® 1135); octadecyl 3-(3,5-di-tert-butyl-4-hydroxyphenyl)propionate (IRGANOX® 1076); 3,7-bis(1,1,3,3-tetramethylbutyl)-10H-phenothiazine (IRGANOX® LO 3); 2,2′-methylenebis(4-methyl-6-tert-butylphenol)monoacrylate (IRGANOX® 3052); 2-tert-butyl-6-[1-(3-tert-butyl-2-hydroxy-5-methylphenyl)ethyl]-4-methylphenyl acrylate (SUMILIZER® TM 4039); 2-[1-(2-hydroxy-3,5-di-tert-pentylphenyl)ethyl]-4,6-di-tert-pentylphenyl acrylate (SUMILIZER® GS); 1,3-dihydro-2H-Benzimidazole (SUMILIZER® MB); 2-methyl-4,6-bis[(octylthio)methyl]phenol (IRGANOX® 1520); N, N′-trimethylenebis-[3-(3,5-di-tert-butyl-4-hydroxyphenyl)propionamide (IRGANOX® 1019); 4-n-octadecyloxy-2,6-diphenylphenol (Irganox® 1063); 2,2′-ethylidenebis[4,6-di-tert-butylphenol] (IRGANOX® 129); N N′-hexamethylenebis(3,5-di-tert-butyl-4-hydroxyhydrocinnamamide) (IRGANOX® 1098); diethyl (3,5-di-tert-butyl-4-hydroxybenxyl)phosphonate (IRGANOX® 1222); 4,4′-di-tert-octyldiphenylamine (Irganox®5057); N-phenyl-1-napthalenamine (IRGANOX® L 05); tris[2-tert-butyl-4-(3-ter-butyl-4-hydroxy-6-methylphenylthio)-5-methyl phenyl]phosphite (HOSTANOX® OSP 1); zinc dinonyidithiocarbamate (HOSTANOX® VP-ZNCS 1); 3,9-bis[1,1-diimethyl-2-[(3-tert-butyl-4-hydroxy-5-methylphenyl)propionyloxy]ethyl]-2,4,8,10-tetraoxaspiro[5.5]undecane (SUMILIZER® AG80); pentaerythrityl tetrakis[3-(3,5-di-tert-butyl-4-hydroxyphenyl)propionate] (IRGANOX® 1010); ethylene-bis(oxyethylene)bis[3-(5-tert-butyl-4-hydroxy-m-tolyl)-propionate (IRGANOX® 245); 3,5-di-tert-butyl-4-hydroxytoluene (Lowinox BHT, Chemtura), the like, and any combination thereof.

Free radical scavengers may be present in the aqueous formaldehyde solutions of the present disclosure at 250 ppm or less (e.g., 200 ppm or less, 150 ppm or less, 100 ppm or less, 75 ppm or less, 50 ppm or less, 25 ppm or less, 0.03 ppm to 250 ppm, 0.03 ppm to 100 ppm, 0.03 ppm to 50 ppm, 0.03 ppm to 25 ppm, 50 ppm to 250 ppm, 50 ppm to 200 ppm, 50 ppm to 150 ppm, or 50 ppm to 100 ppm), based on the total weight of the aqueous formaldehyde solution. More than one free radical scavenger may be used in an aqueous formaldehyde solution of the present disclosure where a cumulative concentration of free radical scavengers is within the foregoing values.

Generally, the concentration of the free radical scavengers in the aqueous formaldehyde solutions of the present disclosure may be higher at higher formaldehyde concentrations. However, free radical scavengers like TEMPO, TEMPOL, and sterically hindered phenolic antioxidant may be effective at lower concentrations of 100 ppm or less (e.g., 50 ppm or less, 25 ppm or less, 0.03 ppm to 100 ppm, 0.03 ppm to 50 ppm, or 0.03 ppm to 25 ppm) with higher concentrations of formaldehyde (e.g., 35 wt % or greater, 40 wt % or greater, 45 wt % or greater, or 50 wt % or greater). Without being limited by theory, said antioxidants, especially TEMPO and TEMPOL, may be self-regenerating, which allows for efficacy at lower concentrations.

In some instances, aqueous formaldehyde solutions of the present disclosure may include 40 wt % or greater of water, 35 wt % or greater of formaldehyde, and 100 ppm or less of a free radical scavenger, each concentration based on a total weight of the aqueous formaldehyde solution. In some instances, aqueous formaldehyde solutions of the present disclosure may include 40 wt % or greater of water, 35 wt % or greater of formaldehyde, and 50 ppm or less of a free radical scavenger, each concentration based on a total weight of the aqueous formaldehyde solution. In some instances, aqueous formaldehyde solutions of the present disclosure may include 40 wt % or greater of water, 40 wt % or greater of formaldehyde, and 50 ppm or less of a free radical scavenger, each concentration based on a total weight of the aqueous formaldehyde solution. In some instances, aqueous formaldehyde solutions of the present disclosure may include 40 wt % or greater of water, 45 wt % or greater of formaldehyde, and 50 ppm or less of a free radical scavenger, each concentration based on a total weight of the aqueous formaldehyde solution. In some instances, aqueous formaldehyde solutions of the present disclosure may include 40 wt % or greater of water, 50 wt % or greater of formaldehyde, and 50 ppm or less of a free radical scavenger, each concentration based on a total weight of the aqueous formaldehyde solution. In some instances, aqueous formaldehyde solutions of the present disclosure may include 40 wt % or greater of water, 35 wt % or greater of formaldehyde, and 25 ppm or less of a free radical scavenger, each concentration based on a total weight of the aqueous formaldehyde solution. In some instances, aqueous formaldehyde solutions of the present disclosure may include 40 wt % or greater of water, 40 wt % or greater of formaldehyde, and 25 ppm or less of a free radical scavenger, each concentration based on a total weight of the aqueous formaldehyde solution. In some instances, aqueous formaldehyde solutions of the present disclosure may include 40 wt % or greater of water, 45 wt % or greater of formaldehyde, and 25 ppm or less of a free radical scavenger, each concentration based on a total weight of the aqueous formaldehyde solution. In some instances, aqueous formaldehyde solutions of the present disclosure may include 40 wt % or greater of water, 50 wt % or greater of formaldehyde, and 25 ppm or less of a free radical scavenger, each concentration based on a total weight of the aqueous formaldehyde solution. In the foregoing examples, the free radical scavenger may preferably include TEMPO, TEMPOL, a sterically hindered phenolic antioxidant, or any combination thereof.

The aqueous formaldehyde solutions of the present disclosure optionally also include other stabilizers like alcohols (e.g., methanol, ethanol, or a combination thereof). When present, said other stabilizers may be present in the aqueous formaldehyde solutions of the present disclosure at 20 wt % or less (e.g., 15 wt % or less, 10 wt % or less, 5 wt % or less, 1 wt % to 20 wt %, 1 wt % to 10 wt %, 1 wt % to 7 wt %, or 1 wt % to 5 wt %), based on the total weight of the aqueous formaldehyde solution.

### Methods

Methods of the present disclosure may include producing aqueous formaldehyde solutions stabilized with free radical scavengers. For example, methanol may be used as a feed in oxidation, oxidative dehydrogenation, or non-oxidative dehydrogenation reaction to produce formaldehyde. The crude product may be treated (e.g., cooled, purified, and the like) to produce an aqueous formaldehyde solution. A free radical scavenger may be added at any point (or at multiple points) after the reaction to produce formaldehyde. More specifically, the free radical scavenger can be added to the product, the aqueous formaldehyde solution, any intermediate fluid therebetween, or any combination thereof. The free radical scavenger may be added per the foregoing so that the resultant aqueous formaldehyde solution has a free radical scavenger concentration 250 ppm or less (or any other concentration per the compositions disclosed herein), based on the total weight of the aqueous formaldehyde solution. Preferably, when the free radical scavenger is added to one of the fluids the temperature of said fluid is below 200° F. to mitigate decomposition of the free radical scavenger.

Any suitable reactant concentrations, reaction conditions, and catalysts may be used producing formaldehyde from methanol and oxygen. Example disclosures for producing formaldehyde may include U.S. Pat. Nos. 3,137,736; 3,532,756; 4,358,623; 4,386,014; 4,450,301; 4,390,730; 6,448,448; 9,120,743; and 12,202,793, each of which is incorporated herein by reference.

Methods of the present disclosure may include storing aqueous formaldehyde solutions stabilized with free radical scavengers.

Aqueous formaldehyde solutions stabilized with free radical scavengers may be stored at temperatures of 80° F. or greater (e.g., 90° F. or greater, 100° F. or greater, 110° F. or greater, 120° F. or greater, 130° F. or greater, 140° F. or greater, 80° F. to 160° F., 100° F. to 160° F., 120° F. to 160° F., 130° F. to 160° F., or 140° F. to 160° F.).

Aqueous formaldehyde solutions stabilized with free radical scavengers may be stored for at least 5 days (e.g., least 10 days, least 30 days, least 45 days, least 60 days, least 80 days, or longer).

In some instances, after storage for 10 days (e.g., 20 days, 30 days, 40 days, or 50 days) at 80° F. or greater (e.g., 90° F. or greater, 100° F. or greater, 110° F. or greater, 120° F. or greater, 130° F. or greater, 140° F. or greater, 80° F. to 160° F., 100° F. to 160° F., 120° F. to 160° F., 130° F. to 160° F., or 140° F. to 160° F.), the aqueous formaldehyde solution may have a formic acid concentration of 1000 ppm or less (e.g., 750 ppm or less, 500 ppm or less, 250 ppm or less, or 100 ppm or less).

The degree of stabilization of the aqueous formaldehyde solutions of the present disclosure may be characterized by the amount of formic acid produced when stored at 140° F. over a specified time starting from when the free radical scavenger was added.

The aqueous formaldehyde solutions of the present disclosure may have a stability characterized by having a formic acid concentration of 1000 ppm or less (e.g., 750 ppm or less, 500 ppm or less, 250 ppm or less, or 100 ppm or less) when stored at 140° F. over 10 days after addition of the free radical scavenger. Preferably, the aqueous formaldehyde solutions of the present disclosure may have a stability characterized by having a formic acid concentration of 500 ppm or less (e.g., 250 ppm or less, or 100 ppm or less) when stored at 140° F. over 10 days after addition of the free radical scavenger.

The aqueous formaldehyde solutions of the present disclosure may have a stability characterized by having a formic acid concentration of 1000 ppm or less (e.g., 750 ppm or less, 500 ppm or less, 250 ppm or less, or 100 ppm or less) when stored at 140° F. over 20 days after addition of the free radical scavenger. Preferably, the aqueous formaldehyde solutions of the present disclosure may have a stability characterized by having a formic acid concentration of 500 ppm or less (e.g., 250 ppm or less, or 100 ppm or less) when stored at 140° F. over 20 days after addition of the free radical scavenger.

The aqueous formaldehyde solutions of the present disclosure may have a stability characterized by having a formic acid concentration of 1000 ppm or less (e.g., 750 ppm or less, 500 ppm or less, 250 ppm or less, or 100 ppm or less) when stored at 140° F. over 40 days after addition of the free radical scavenger. Preferably, the aqueous formaldehyde solutions of the present disclosure may have a stability characterized by having a formic acid concentration of 500 ppm or less (e.g., 400 ppm or less, 300 ppm or less, 250 ppm or less, or 200 ppm or less) when stored at 140° F. over 40 days after addition of the free radical scavenger.

The degree of stabilization of the aqueous formaldehyde solutions of the present disclosure may be characterized by the amount of formic acid produced when stored at 140° F. over a specified time starting from when the free radical scavenger was added compared to the amount of formic acid produced when a comparable aqueous formaldehyde solution (i.e., an aqueous formaldehyde solution having the same composition but without the free radical scavenger) stored at 140° F. over a specified time.

The aqueous formaldehyde solutions of the present disclosure may have a stability characterized by having a formic acid concentration of at least 50 ppm lower than (e.g., at least 100 ppm lower than, at least 200 ppm lower than, or at least 300 ppm lower than) a formic acid concentration in a comparable aqueous formaldehyde solution when each re stored at 140° F. over 10 days. Preferably, aqueous formaldehyde solutions of the present disclosure may have a stability characterized by having a formic acid concentration of at least 100 ppm lower than (e.g., at least 200 ppm lower than, or at least 300 ppm lower than) a formic acid concentration in a comparable aqueous formaldehyde solution when each re stored at 140° F. over 10 days.

The aqueous formaldehyde solutions of the present disclosure may have a stability characterized by having a formic acid concentration of at least 100 ppm lower than (e.g., at least 200 ppm lower than, at least 300 ppm lower than, or at least 400 ppm lower than) a formic acid concentration in a comparable aqueous formaldehyde solution when each re stored at 140° F. over 20 days. Preferably, aqueous formaldehyde solutions of the present disclosure may have a stability characterized by having a formic acid concentration of at least 200 ppm lower than (e.g., at least 300 ppm lower than, or at least 400 ppm lower than) a formic acid concentration in a comparable aqueous formaldehyde solution when each re stored at 140° F. over 20 days.

The aqueous formaldehyde solutions of the present disclosure may have a stability characterized by having a formic acid concentration of at least 300 ppm lower than (e.g., at least 400 ppm lower than, at least 500 ppm lower than, or at least 600 ppm lower than) a formic acid concentration in a comparable aqueous formaldehyde solution when each re stored at 140° F. over 40 days. Preferably, aqueous formaldehyde solutions of the present disclosure may have a stability characterized by having a formic acid concentration of at least 500 ppm lower than (e.g., at least 600 ppm lower than, at least 700 ppm lower than, or at least 800 ppm lower than) a formic acid concentration in a comparable aqueous formaldehyde solution when each re stored at 140° F. over 40 days.

The following numbered embodiments are contemplated. All combinations of features and embodiments are contemplated.

Embodiment 1. An aqueous formaldehyde solution comprising: 40 wt % or greater of water; 30 wt % or greater of formaldehyde; and 250 ppm or less of a free radical scavenger, each concentration based on a total weight of the aqueous formaldehyde solution.

Embodiment 2. The aqueous formaldehyde solution of Embodiment 1, wherein the free radical scavenger comprises 2,2,6,6-tetramethyl-1-piperidiny-1-oxyl; 4-hydroxy-2,2,6,6-tetramethyl-1-piperidin-1-oxyl; a sterically hindered phenolic antioxidant; copper; 3,7-bis(dimethylamino)-phenothiazin-5-ium chloride; pentylenetetrazol; or any combination thereof.

Embodiment 3. The aqueous formaldehyde solution of any preceding Embodiment, wherein the free radical scavenger is present at 200 ppm or less.

Embodiment 4. The aqueous formaldehyde solution of any preceding Embodiment, wherein the formaldehyde is present at 35 wt % or greater and the free radical scavenger is present at 50 ppm or less.

Embodiment 5. The aqueous formaldehyde solution of Embodiment 4, wherein the free radical scavenger comprises 2,2,6,6-tetramethyl-1-piperidiny-1-oxyl; 4-hydroxy-2,2,6,6-tetramethyl-1-piperidin-1-oxyl; a sterically hindered phenolic antioxidant; or any combination thereof.

Embodiment 6. The aqueous formaldehyde solution of any preceding Embodiment, wherein the formaldehyde is present at 50 wt % or greater.

Embodiment 7. The aqueous formaldehyde solution of any preceding Embodiment, further comprising: 1 wt % to 20 wt % of methanol, 1 wt % to 20 wt % of ethanol, or 1 wt % to 20 wt % of a combination of methanol and ethanol.

Embodiment 8. A method comprising: producing a product comprising formaldehyde from methanol; treating the product to produce an aqueous formaldehyde solution comprising 40 wt % or greater of water and 30 wt % or greater of formaldehyde, each concentration based on a total weight of the aqueous formaldehyde solution; and adding a free radical scavenger to the product, the aqueous formaldehyde solution, any intermediate fluid therebetween, or any combination thereof such that a concentration of the free radical scavenger in the aqueous formaldehyde solution is 250 ppm or less, based on the total weight of the aqueous formaldehyde solution.

Embodiment 9. The method of Embodiment 8, wherein the fluid to which the free radical scavenger is added has a temperature of 200° F. or less.

Embodiment 10. The method of any one of Embodiments 8-9, wherein the free radical scavenger comprises 2,2,6,6-tetramethyl-1-piperidiny-1-oxyl; 4-hydroxy-2,2,6,6-tetramethyl-1-piperidin-1-oxyl; a sterically hindered phenolic antioxidant; copper; 3,7-bis(dimethylamino)-phenothiazin-5-ium chloride; pentylenetetrazol; or any combination thereof.

Embodiment 11. The method of any one of Embodiments 8-10, wherein the free radical scavenger is present at 200 ppm or less.

Embodiment 12. The method of any one of Embodiments 8-11, wherein the formaldehyde is present at 35 wt % or greater and the free radical scavenger is present at 50 ppm or less.

Embodiment 13. The method of Embodiment 12, wherein the free radical scavenger comprises 2,2,6,6-tetramethyl-1-piperidiny-1-oxyl; 4-hydroxy-2,2,6,6-tetramethyl-1-piperidin-1-oxyl; a sterically hindered phenolic antioxidant; or any combination thereof.

Embodiment 14. The method of any one of Embodiments 8-13, wherein the formaldehyde is present at 50 wt % or greater.

Embodiment 15. The method of any one of Embodiments 8-14, wherein the aqueous formaldehyde solution after adding the free radical scavenger is characterized by having a formic acid concentration of 1000 ppm or less when stored at 140° C. for 10 days after addition of the free radical scavenger.

Embodiment 16. The method of any one of Embodiments 8-15, wherein the aqueous formaldehyde solution after adding the free radical scavenger is characterized by having a formic acid concentration of at least 50 ppm lower than a concentration of formic acid in a comparable aqueous formaldehyde solution, each stored at 140° C. for 10 days.

Embodiment 17. A method comprising: storing an aqueous formaldehyde solution at 80° F. or greater, the aqueous formaldehyde solution comprising 40 wt % or greater of water, 30 wt % or greater of formaldehyde, and 250 ppm or less of a free radical scavenger, each concentration based on a total weight of the aqueous formaldehyde solution.

Embodiment 18. The method of Embodiment 17, wherein after storing for 10 days the aqueous formaldehyde solution has a formic acid concentration of 1000 ppm or less.

Embodiment 19. The method of any one of Embodiments 17-18, wherein after storing for 30 days the aqueous formaldehyde solution has a formic acid concentration of 500 ppm or less.

Embodiment 20. The method of any one of Embodiments 17-19, wherein the aqueous formaldehyde solution is stored at 120° F. or greater.

### EXAMPLES

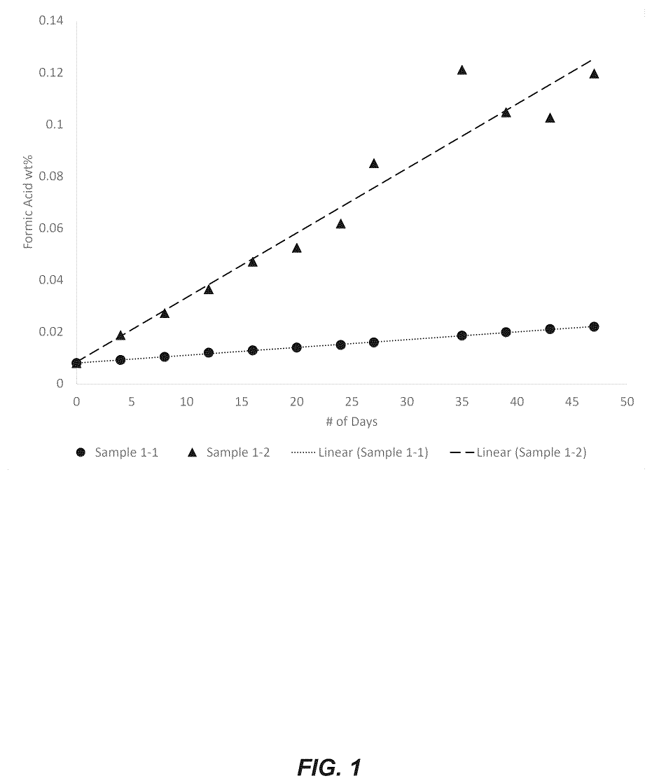

Example 1. An aqueous formaldehyde solution (Sample 1-1) was prepared containing 50 wt % formaldehyde, 25 ppm TEMPO, and a balance of water. A comparative sample (Sample 1-2) was prepared containing 50 wt % formaldehyde, no TEMPO, and a balance of water. The samples were heated and stored at 140° F. for 47 days. During storage, the concentration of formic acid in each sample was tested. FIG. 1 is a plot of the concentration of formic acid in each of the samples over time.

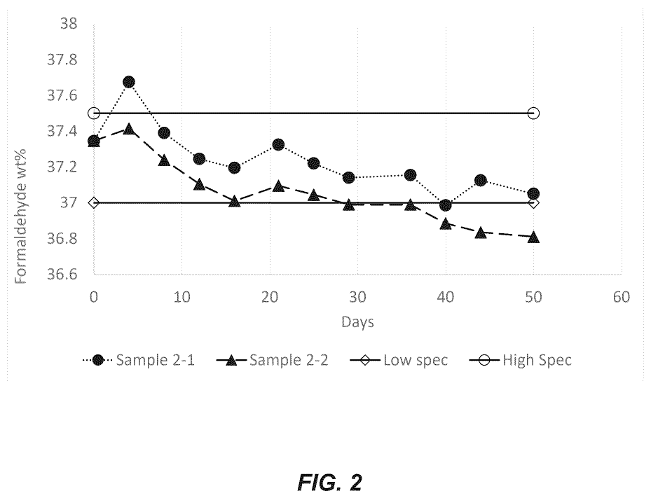

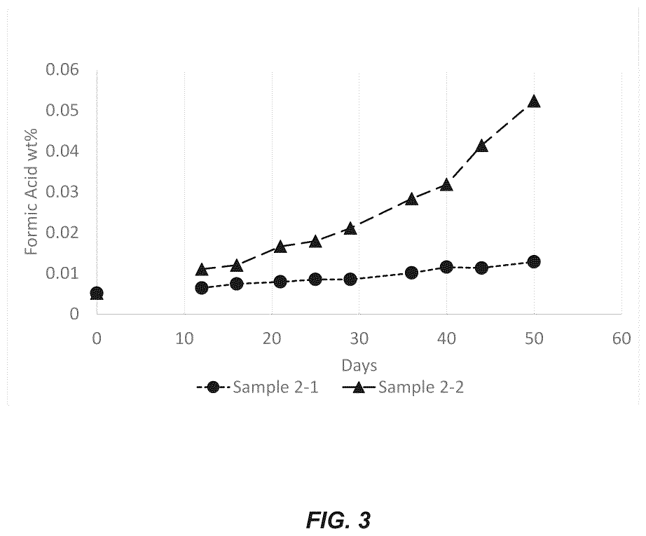

Example 2. An aqueous formaldehyde solution (Sample 2-1) was prepared containing 37 wt % formaldehyde, 7 wt % methanol, 25 ppm TEMPO, and a balance of water. A comparative sample (Sample 2-2) was prepared containing 37 wt % formaldehyde, 7 wt % methanol, no TEMPO, and a balance of water. The samples were stored at 130° F. for 50 days. During storage, the concentrations of formaldehyde and formic acid in each sample were tested. FIG. 2 is a plot of the concentration of formaldehyde in each of the samples over time. The specification for 37 wt % formaldehyde in this example is to have a formaldehyde concentration from 37.0 wt % to 37.5 wt %, which is overlaid on FIG. 2. FIG. 3 is a plot of the concentration of formic acid in each of the samples over time.

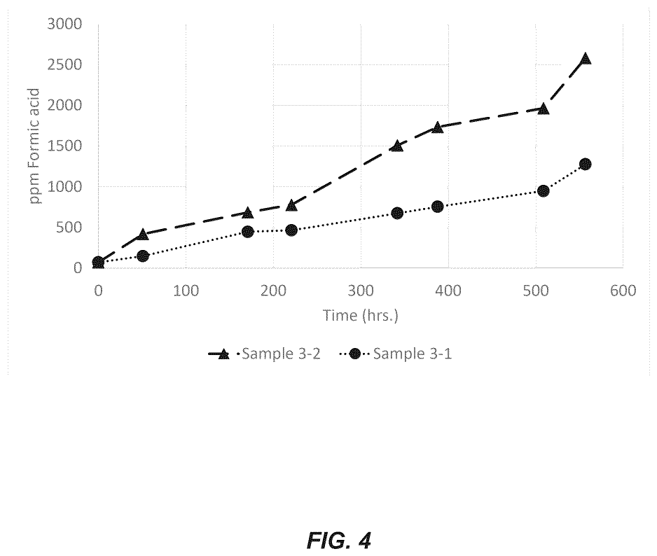

Example 3. An aqueous formaldehyde solution (Sample 3-1) was prepared containing 50 wt % formaldehyde, 100 ppm TEMPO, and a balance of water. A comparative sample (Sample 3-2) was prepared containing 50 wt % formaldehyde, no TEMPO, and a balance of water. The samples were stored at 185° F. for 23 days. During storage, the concentrations of formaldehyde and formic acid in each sample were tested. FIG. 4 is a plot of the concentration of formic acid in each of the samples over time. This experiment illustrates that TEMPO is effective at temperatures approaching the degradation temperature of TEMPO, about 200° F.

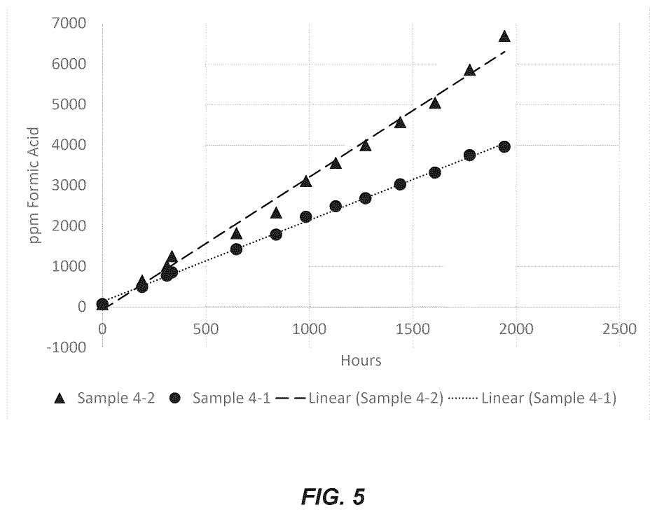

Example 4. An aqueous formaldehyde solution (Sample 4-1) was prepared containing 50 wt % formaldehyde, 150 ppm methylene blue, and a balance of water. A comparative sample (Sample 4-2) was prepared containing 50 wt % formaldehyde, no methylene blue, and a balance of water. The samples were stored at 185° F. for 23 days. During storage, the concentration of formic acid in each sample was tested. FIG. 5 is a plot of the concentration of formic acid in each of the samples over time.

Example 5. An aqueous formaldehyde solution (Sample 5-1) was prepared containing 50 wt % formaldehyde, 100 ppm PTZ, and a balance of water. A comparative sample (Sample 5-2) was prepared containing 50 wt % formaldehyde, no PTZ, and a balance of water. The samples were stored at 185° F. After about 5 days, Sample 5-1 contained 319 ppm formic acid, while Sample 5-2 contained 449 ppm formic acid.

Example 6. An aqueous formaldehyde solution (Sample 6-1) was prepared containing 50 wt % formaldehyde, 150 ppm IRGANOX® 259, and a balance of water. A comparative sample (Sample 6-2) was prepared containing 50 wt % formaldehyde, no IRGANOX® 529, and a balance of water. The samples were stored at 185° F. After about 7 days, Sample 6-1 contained 621 ppm formic acid, while Sample 6-2 contained 886 ppm formic acid. After about 11.7 days, Sample 6-1 contained 887 ppm formic acid, while Sample 6-2 contained 1112 ppm formic acid.

Example 7. An aqueous formaldehyde solution (Sample 7-1) was prepared containing 50 wt % formaldehyde, 25 ppm TEMPO, and a balance of water. A comparative sample (Sample 7-2) was prepared containing 50 wt % formaldehyde, no TEMPO, and a balance of water. The samples were stored at heated to 160° F. and each day for 24 days the temperature was dropped 2° F. During storage, the samples were observed for visible signs of paraformaldehyde formation. At the end of 24 days, the concentration of formic acid in each sample was measured.

At day 14, both samples were clear but precipitated paraformaldehyde was observed in each sample. The amount of precipitate was less for Sample 7-1. At day 23, which was at 114° F., Sample 7-2 was hazy due to sufficient paraformaldehyde formation and precipitation. At day 24,which was at 112° F., Sample 7-1 became hazy but, upon swirling the sample, the paraformaldehyde precipitates were not stuck to the walls of the container. In contrast, sufficient amounts of paraformaldehyde was precipitated in Sample 7-2 to be stuck to the container wall. At day 24, Sample 7-1 contained 179 ppm of formic acid, and Sample 7-2 contained 640 ppm of formic acid. From production to use including shipping and storage, the temperature of the formaldehyde typically drops about 2° F. per day, which may change based on the location of shipping and storage. This example illustrates that TEMPO allows for longer storage and transport times before visible formation of paraformaldehyde.

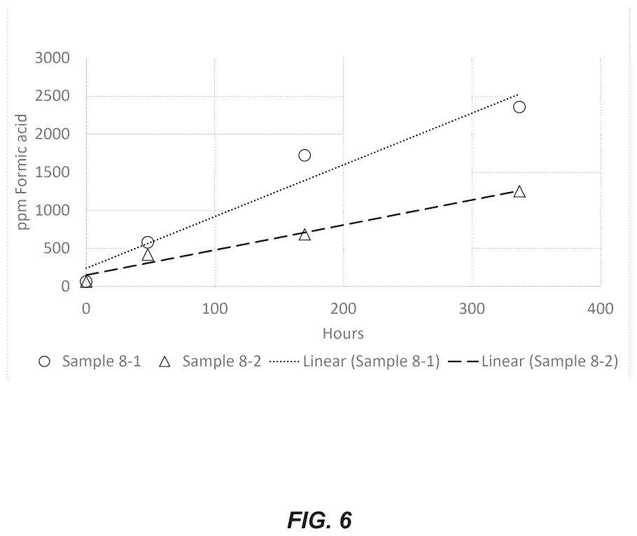

Example 8. An aqueous formaldehyde solution (Sample 8-1) was prepared containing 50 wt % formaldehyde, 200 ppm azobisisobutyronitrile (AIBN), and a balance of water. A comparative sample (Sample 8-2) was prepared containing 50 wt % formaldehyde, no AIBN, and a balance of water. AIBN is a free radical initiator. The samples were stored at 185° F. During storage, the concentrations of formic acid in each sample was tested. FIG. 6 is a plot of the concentration of formic acid in each of the samples over time. The inclusion of a free radical initiator (i.e., increasing the concentration of free radicals) in the aqueous formaldehyde solution increased the amount of formic acid produced over time.

While the invention has been described in detail, modifications within the spirit and scope of the invention will be readily apparent to those of skill in the art. It should be understood that aspects of the invention and portions of various embodiments and various features recited herein and/or in the appended claims may be combined or interchanged either in whole or in part. In the foregoing descriptions of the various embodiments, those embodiments which refer to another embodiment may be appropriately combined with other embodiments as will be appreciated by one of ordinary skill in the art. Furthermore, those of ordinary skill in the art will appreciate that the foregoing description is by way of example only, and is not intended to limit the invention.

We claim:

1. An aqueous formaldehyde solution comprising:
40 wt % or greater of water;
30 wt % or greater of formaldehyde; and
0.03 ppm to 250 ppm of a free radical scavenger, each concentration based on a total weight of the aqueous formaldehyde solution.

2. The aqueous formaldehyde solution of claim 1, wherein the free radical scavenger comprises 2,2,6,6-tetramethyl-1-piperidiny-1-oxyl; 4-hydroxy-2,2,6,6-tetramethyl-1-piperidin-1-oxyl; a sterically hindered phenolic antioxidant; copper; 3,7-bis(dimethylamino)-phenothiazin-5-ium chloride; pentylenetetrazol; or any combination thereof.

3. The aqueous formaldehyde solution of claim 1, wherein the free radical scavenger is present at 0.03 ppm to 200 ppm.

4. The aqueous formaldehyde solution of claim 1, wherein the formaldehyde is present at 35 wt % or greater and the free radical scavenger is present at 0.03 ppm to 50 ppm.

5. The aqueous formaldehyde solution of claim 4, wherein the free radical scavenger comprises 2,2,6,6-tetramethyl-1-piperidiny-1-oxyl; 4-hydroxy-2,2,6,6-tetramethyl-1-piperidin-1-oxyl; a sterically hindered phenolic antioxidant; or any combination thereof.

6. The aqueous formaldehyde solution of claim 1, wherein the formaldehyde is present at 50 wt % or greater.

7. The aqueous formaldehyde solution of claim 1, further comprising: 1 wt % to 20 wt % of methanol, 1 wt % to 20 wt % of ethanol, or 1 wt % to 20 wt % of a combination of methanol and ethanol.

8. A method comprising:
producing a product comprising formaldehyde from methanol;
treating the product to produce an aqueous formaldehyde solution comprising 40 wt % or greater of water and 30 wt % or greater of formaldehyde, each concentration based on a total weight of the aqueous formaldehyde solution; and
adding a free radical scavenger to the product, the aqueous formaldehyde solution, any intermediate fluid therebetween, or any combination thereof such that a concentration of the free radical scavenger in the aqueous formaldehyde solution is 0.03 ppm to 250 ppm, based on the total weight of the aqueous formaldehyde solution.

9. The method of claim 8, wherein the fluid to which the free radical scavenger is added has a temperature of 200° F. or less.

10. The method of claim 8, wherein the free radical scavenger comprises 2,2,6,6-tetramethyl-1-piperidiny-1-oxyl;
4-hydroxy-2,2,6,6-tetramethyl-1-piperidin-1-oxyl; a sterically hindered phenolic antioxidant; copper; 3,7-bis(dimethylamino)-phenothiazin-5-ium chloride;
pentylenetetrazol; or any combination thereof.

11. The method of claim 8, wherein the free radical scavenger is present at 0.03 ppm to 200 ppm.

12. The method of claim 8, wherein the formaldehyde is present at 35 wt % or greater and the free radical scavenger is present at 0.03 ppm to 50 ppm.

13. The method of claim 12, wherein the free radical scavenger comprises 2,2,6,6-tetramethyl-1-piperidiny-1-oxyl;
4-hydroxy-2,2,6,6-tetramethyl-1-piperidin-1-oxyl; a sterically hindered phenolic antioxidant; or any combination thereof.

14. The method of claim 8, wherein the formaldehyde is present at 50 wt % or greater.

15. The method of claim 8, wherein the aqueous formaldehyde solution after adding the free radical scavenger is characterized by having a formic acid concentration of 1000 ppm or less when stored at 140° C. for 10 days after addition of the free radical scavenger.

16. The method of claim 8, wherein the aqueous formaldehyde solution after adding the free radical scavenger is characterized by having a formic acid concentration of at least 50 ppm lower than a concentration of formic acid in a comparable aqueous formaldehyde solution, each stored at 140° C. for 10 days.

17. A method comprising:
storing an aqueous formaldehyde solution at 80° F. or greater, the aqueous formaldehyde solution comprising 40 wt % or greater of water, 30 wt % or greater of formaldehyde, and 0.03 ppm to 250 ppm of a free radical scavenger, each concentration based on a total weight of the aqueous formaldehyde solution.

18. The method of claim 17, wherein after storing for 10 days the aqueous formaldehyde solution has a formic acid concentration of 1000 ppm or less.

19. The method of claim 17, wherein after storing for 30 days the aqueous formaldehyde solution has a formic acid concentration of 500 ppm or less.

20. The method of claim 17, wherein the aqueous formaldehyde solution is stored at 120° F. or greater.
